(19) United States
(12) Patent Application Publication
(10) Pub. No.: US 2025/0382661 A1
(43) Pub. Date: Dec. 18, 2025

(54) METHODS AND COMPOSITIONS TO DETECT CHEMICAL ADDUCTS ON OLIGONUCLEOTIDES

(71) Applicant: THE REGENTS OF THE UNIVERSITY OF CALIFORNIA, Oakland, CA (US)

(72) Inventors: Robert Spitale, Irvine, CA (US); Natalie Falco, Irvine, CA (US); Chely M. Garfio, Irvine, CA (US); Leslie Spitalny, Irvine, CA (US)

(21) Appl. No.: 18/878,493

(22) PCT Filed: Jun. 30, 2023
(86) PCT No.: PCT/US23/69500

§ 371 Date: Dec. 23, 2024

Related U.S. Application Data
(60) Provisional application No. 63/357,246, filed on Jun. 30, 2022.

(51) Int. Cl. C12Q 1/6818

(52) U.S. Cl. CPC C12Q 1/6818

(57) ABSTRACT

A reverse transcription (RT) assay to directly detect chemical adducts on RNA. A fluorescence quenching assay to detect RT polymerization was optimized and employed to detect N 1-alkylation of inosine, an important post-transcriptional modification, using a phenylacrylamide as a model compound. The methods and composition may be expanded to identify novel reagents that form adducts with RNA, regardless of the primary sequence, and further explored to understand the relationship between RT processivity and natural post-transcriptional modifications in RNA.

### CROSS-REFERENCES TO RELATED APPLICATIONS

This application claims benefit of U.S. Provisional Application No. 63/357,246 filed Jun. 30, 2022, the specification of which is incorporated herein in their entirety by reference.

### FIELD OF THE INVENTION

The present invention features systems and methods for detecting a potential adduct on a target oligonucleotide. In a non-limiting embodiment, the present invention features a fluorescent reverse-transcription assay to detect chemical adducts on RNA.

### BACKGROUND OF THE INVENTION

RNA molecules perform key functions at the heart of many biological pathways and are significant drivers in the onset of many diseases. RNA molecules are also prone to modifications, many of which have been characterized to control RNA structure, function, and RNA-protein interactions. In addition, researchers have a growing interest in identifying RNA-small molecule interactions, with a specific focus on discovering small molecules that can bind to RNA and introduce ligand-dependent covalent adducts. Subsequently, there is a rising demand for the development of RNA-centric assays to directly detect chemical interactions or modifications on RNA.

Several biochemical assays have been developed in order to identify RNA-ligand interactions of post-transcriptional chemical modifications. NMR and other spectroscopic techniques can report binding with high resolution but have severely limited scalability. Chemical microarrays have proven their utility in screening a desired RNA for binding, but the chemical space is limited due to the available functional groups that are compatible with arrays. Additionally, fluorescent nucleotides and fluorescent ligand displacement assays have limited scope because they only report on a predetermined site in a primary RNA sequence. While these approaches are useful for identifying ligand-RNA interactions; they do not directly report covalent RNA adducts. The ongoing demand to exploit covalent modifications for functional control of biomolecules requires the development of systems and methods for the direct detection of RNA adducts.

### BRIEF SUMMARY OF THE INVENTION

It is an objective of the present invention to provide systems and methods that can detect chemical adducts on a target oligonucleotide, as specified in the independent claims. Embodiments of the invention are given in the dependent claims. Embodiments of the present invention can be freely combined with each other if they are not mutually exclusive.

In some embodiments, the present invention features a method of detecting a potential adduct on a target oligonucleotide. The method further comprises conjugating a fluorophore to the target oligonucleotide, where the fluorophore is downstream of the potential adduct to be detected, and reverse transcribing (RT) the target oligonucleotide upstream of the potential adduct to form an RT-extension. If no adduct exists on the target oligonucleotide, then the RT-extension quenches the fluorophore's fluorescence. If an adduct exists on the target oligonucleotide, then the RT-extension is truncated and the fluorophore's fluorescence is maintained.

The first goal in developing an approach to detect chemical adducts on RNA was to utilize the unique characteristics of RT assays. A recent RT-based approach was published, but it relied on costly RNA sequencing to determine the site of adduct formation. Instead, the present invention sought to develop a fluorescence protocol that could easily be performed in a lab setting using a more conventional readout. The disclosed method can be used for any RNA, regardless of the primary sequence.

In other embodiments, the present invention features a system for detecting an adduct on a target oligonucleotide. The system comprises a target oligonucleotide labeled with a fluorophore downstream of a potential adduct to be detected; and a reverse transcribing (RT) component for acting upstream of the potential adduct. The RT component can reverse transcribe the target oligonucleotide to form an RT-extension. If no adduct exists on the target oligonucleotide, then the RT-extension quenches the fluorophore's fluorescence. If an adduct exists on the target oligonucleotide, then the RT-extension is truncated and the fluorophore's fluorescence is maintained.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a schematic of a Fluorescence RT Assay. In the present study, a fluorescence RT assay has been developed wherein the absence of an adduct on a fluorophore-conjugated oligonucleotide allows RT to occur. If full RT elongation occurs, fluorescence is attenuated. If an adduct is present on the oligonucleotide, RT is unable to proceed, and the fluorescence signal remains. In this proof-of-concept, an internal C18 spacer and a clicked phenyl acrylamide molecule are shown to stop RT elongation and prevent fluorescence attenuation.

FIGS. 2A, 2B, 2C, 2D, and 2E show the development of a fluorescence RT assay using DNA oligonucleotides. FIG. 2A shows chemical structures of the fluorophores used in the present study. FIG. 2B shows a schematic of the hybridization experiment where antisense-annealing quenches fluorescence. FIG. 2C shows the quantification of the percent quenching of annealed DNA oligonucleotide with their respective fluorophores. Error bars represent the SD of n=3. FIG. 2D shows the quantification of the percent quenching of annealed BDP FL-DNA oligonucleotide with different linker lengths. Error bars represent the SD of n=3. FIG. 2E shows raw spectral data of the experiment quantified in FIG. 2D.

FIGS. 3A, 3B, 3C, 3D, 3E, and 3F show development of a fluorescence RT Assay using RNA Oligonucleotides. FIG. 3A shows a schematic of the RT experiment wherein the absence of an adduct on the RNA oligo-nucleotide allows RT to proceed thereby attenuating fluorescence intensity. Schematic of an RNA oligonucleotide that contains an internal C18 spacer inhibiting RT processivity and maintaining fluorescence. FIG. 3B shows the quantification of the percent quenching of a fluorophore-conjugated RNA oligonucleotide that has undergone RT. An RT primer titration was carried out from 1:1 to 8:1 (RT Primer: RNA). RNA with an internal C18 spacer was used in the control lanes. Error bars represent the SD of n=3. FIG. 3C shows a reaction scheme of EPhAA with inosine. FIG. 3D shows a schematic of the RT experiment of when an inosine-containing RNA oligonucleotide is reacted in the presence and absence of EPhAA. In the absence of the EPhAA reaction, RT elongation occurs, attenuating fluorescent intensity, and in the presence of the EPhAA reaction, RT processivity is halted and fluorescent intensity remains. FIG. 3E shows a radioactive gel of the data shown in FIG. 3D. Note that RT stops at the nucleotide preceding the inosine-EPhAA site. FIG. 3F shows an integrated fluorescent intensity of EPhAA incubated inosine-modified RNA oligonucleotides post-RT. EPhAA was incubated with an inosine-modified RNA oligonucleotide at various concentrations in 50:50 EtOH:TEAA at 70° C. for 24 h. Error bars represent the SD of n=3.

FIG. 4A shows a diagram of the secondary structure of the preQ1 riboswitch. In order to employ this riboswitch in our assay, a poly C-tail was installed at the 5′ end and the reverse complement of our primer was installed at the 3′ end.

FIG. 4B shows the quantification of the percent quenching of BDP-preQ1 RNA that has undergone RT relative to its negative control.

FIG. 4C shows a radioactive gel of the data shown in FIG. 4B. The 57th nucleotide corresponds to the full length cDNA.

### DETAILED DESCRIPTION OF THE INVENTION

For purposes of summarizing the disclosure, certain aspects, advantages, and novel features of the disclosure are described herein. It is to be understood that not necessarily all such advantages may be achieved in accordance with any particular embodiment of the disclosure. Thus, the disclosure may be embodied or carried out in a manner that achieves or optimizes one advantage or group of advantages as taught herein without necessarily achieving other advantages as may be taught or suggested herein.

Additionally, although embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. Moreover, while a number of variations have been shown and described in varying detail, other modifications, which are within the scope of the present disclosure, will be readily apparent to those of skill in the art based upon this disclosure. It is also contemplated that various combinations or sub combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described herein.

As used herein, “RT-extension” means an RNA-cDNA hybrid extension.

Referring to the figures, the present invention features a fluorescent reverse-transcription assay to detect chemical adducts on RNA. In some embodiments, the assay comprises a target oligonucleotide labeled with a fluorophore downstream of a potential adduct to be detected, and a reverse transcribing (RT) component for acting upstream of the potential adduct. The RT component reverse can transcribe the target oligonucleotide to form an RT-extension. If no adduct exists on the target oligonucleotide, then the RT-extension quenches the fluorophore's fluorescence. If an adduct exists on the target oligonucleotide, then the RT-extension is truncated and the fluorophore's fluorescence is maintained.

According to some embodiments, the present invention features a method of detecting a potential adduct on a target oligonucleotide. The method further comprises (a) conjugating a fluorophore to the target oligonucleotide, wherein the fluorophore is downstream of the potential adduct to be detected; and (b) reverse transcribing (RT) the target oligonucleotide upstream of the potential adduct; wherein reverse transcribing forms an RT-extension. If no adduct exists on the target oligonucleotide, then the RT-extension quenches the fluorophore's fluorescence. If an adduct exists on the target oligonucleotide, then the RT-extension is truncated and the fluorophore's fluorescence is maintained.

In some embodiments, the fluorophore conjugated to the target oligonucleotide comprises both intrinsic and extrinsic fluorophores that would have their fluorescence altered by their proximity to nucleic acids. In other embodiments, the fluorophore comprises Bodipy fl (BDP-FL), tetramethylrhodamine (TMR), coumarin 343X (C343 X), or a combination thereof.

In some embodiments, the fluorophore is conjugated to the target oligonucleotide via a linker. In preferred embodiments, the linker comprises a chain having 6-12 carbons. In other embodiments, the linker comprises a chain having 0-3 carbons. In some embodiments, the linker comprises a chain having 4-6 carbons. In other embodiments, the linker comprises a chain having 3-15 carbons. In other embodiments, the linker comprises a chain having 6-15 carbons. In some embodiments, the linker comprises a chain having 15 or more carbons. In other embodiments, the linker further comprises one or more non-carbon, including oxygen, sulfur, phosphorus, and/or nitrogen atoms. Non-limiting examples of the potential linker chains are listed in the table below. It is to be understood that the present invention is not to be limited to said examples, and that other examples are within the scope of the invention. Non-limiting examples of the potential linker chains are listed in the table below. It is to be understood that the present invention is not to be limited to said examples, and that other examples are within the scope of the invention.

Non-Limiting Examples of Potential Linker Chains

| | |
|---|---|
| Carbons only | C |
| | C—C |
| | C—C—C |
| | C—C—C—C |
| | C—C—C—C—C |
| | C—C—C—C—C—C |
| | C—C—C—C—C—C—C |
| | C—C—C—C—C—C—C—C |
| | C—C—C—C—C—C—C—C—C |
| | C—C—C—C—C—C—C—C—C—C |
| | C—C—C—C—C—C—C—C—C—C—C |
| | C—C—C—C—C—C—C—C—C—C—C—C |
| | C—C—C—C—C—C—C—C—C—C—C—C—C |
| | C—C—C—C—C—C—C—C—C—C—C—C—C—C |
| | C—C—C—C—C—C—C—C—C—C—C—C—C—C—C |
| | C—C—C—C—C—C—C—C—C—C—C—C—C—C—C—C |
| Carbon and Non-Carbons | Linker chains including carbon, oxygen, |
| | sulfur, phosphorous, and/or nitrogen. |
| Non-Carbon Only | Linker chains including oxygen, sulfur, |
| | phosphorus, and/or nitrogen. |

In some embodiments, the target oligonucleotide comprises RNA or DNA. In other embodiments, the target oligonucleotide further comprises RNA, regardless of the primary sequence. In some embodiments, the target oligonucleotide further comprises single stranded RNA or single stranded DNA. In some embodiments, the target oligonucleotide further comprises single stranded RNA or single stranded DNA capable of forming more complex 2-D or 3-D structures.

In some embodiments, the adduct is a post-translational modification component. For example, the post-translational component may comprise N1-alkylation of inosine. In some embodiments, the method can be used to detect a potential adduct on a target oligonucleotide is used for high throughput screening.

According to other embodiments, the present invention features a system for detecting an adduct on a target oligonucleotide. The system comprises (a) a target oligonucleotide labeled with a fluorophore downstream of a potential adduct to be detected; and (b) a reverse transcribing (RT) component for acting upstream of the potential adduct; wherein the RT component reverse transcribes the target oligonucleotide to form an RT-extension. If no adduct exists on the target oligonucleotide, then the RT-extension quenches the fluorophore's fluorescence. If an adduct exists on the target oligonucleotide, then the RT-extension is truncated and the fluorophore's fluorescence is maintained.

Fluorophores are classified into two main groups: intrinsic and extrinsic. Fluorophores that are naturally obtained are termed as intrinsic, e.g., aromatic amino acids, derivatives of pyridoxal, flavins, NADH, and chlorophyll. In some embodiments, the fluorophore conjugated to the target oligonucleotide comprises both intrinsic and extrinsic fluorophores that would have their fluorescence altered by their proximity to nucleic acids. In other embodiments, the fluorophore comprises Bodipy fl (BDP-FL), tetramethylrhodamine (TMR), coumarin 343X (C343 X), or a combination thereof.

In some embodiments, the fluorophore is conjugated to the target oligonucleotide via a linker. In preferred embodiments, the linker comprises a chain having 6-12 carbons. In other embodiments, the linker comprises a chain having 0-3 carbons. In some embodiments, the linker comprises a chain having 4-6 carbons. In other embodiments, the linker comprises a chain having 3-15 carbons. In other embodiments, the linker comprises a chain having 6-15 carbons. In some embodiments, the linker comprises a chain having 15 or more carbons. In other embodiments, the linker further comprises one or more non-carbon, including oxygen, sulfur, phosphorus, and/or nitrogen. Non-limiting examples of the potential linker chains are listed in the table below. It is to be understood that the present invention is not to be limited to said examples, and that other examples are within the scope of the invention.

Non-Limiting Examples of Potential Linker Chains

| | |
|---|---|
| Carbons only | C |
| | C—C |
| | C—C—C |
| | C—C—C—C |
| | C—C—C—C—C |
| | C—C—C—C—C—C |
| | C—C—C—C—C—C—C |
| | C—C—C—C—C—C—C—C |
| | C—C—C—C—C—C—C—C—C |
| | C—C—C—C—C—C—C—C—C—C |
| | C—C—C—C—C—C—C—C—C—C—C |
| | C—C—C—C—C—C—C—C—C—C—C—C |
| | C—C—C—C—C—C—C—C—C—C—C—C—C |
| | C—C—C—C—C—C—C—C—C—C—C—C—C—C |
| | C—C—C—C—C—C—C—C—C—C—C—C—C—C—C |
| | C—C—C—C—C—C—C—C—C—C—C—C—C—C—C—C |
| Carbon and Non-Carbons | Linker chains including carbon, oxygen, |
| | sulfur, phosphorous, and/or nitrogen. |
| Non-Carbon Only | Linker chains including oxygen, sulfur, |
| | phosphorus, and/or nitrogen. |

In some embodiments, the target oligonucleotide comprises RNA or DNA. In other embodiments, the target oligonucleotide further comprises RNA, regardless of the primary sequence. In some embodiments, the target oligonucleotide further comprises single stranded RNA or single stranded DNA. In some embodiments, the target oligonucleotide further comprises single stranded RNA or single stranded DNA with double stranded character.

In some embodiments, the adduct is a post-translational modification component. For example, the post-translational component may comprise N1-alkylation of inosine. In some embodiments, the system can be used to detect a potential adduct on a target oligonucleotide is used for high throughput screening.

### EXAMPLE

The following is a non-limiting example of the present invention. It is to be understood that said example is not intended to limit the present invention in any way. Equivalents or substitutes are within the scope of the present invention.

The inventors envisioned an assay that could use fluorescence to detect full length RT cDNA and RT truncation at the site of a covalent adduct (FIG. 1). To achieve this, a molecular beacon design was implemented wherein a fluorescence is retained upon RT-truncation, or quenched upon full RNA-cDNA hybrid extension. Working toward this goal, which fluorophore would be ideal for the turn-off system was determined, and then how cDNA hybridization quenching was proximally tied to the conjugation site of the fluorophore on the RNA was established.

DNA molecular beacons are designed to rely on DNA-DNA intramolecular hybridization to control fluorescence and quenching. One part of the single stranded DNA is appended with a fluorophore and the other end a fluorescent quencher. Aiming toward the eventual utility of a RT reaction, the present invention does not rely on attached quencher molecules, but the act of hybridization to quench fluorescence. The physiochemical properties of a variety of fluorophores were tested to understand their quenching capabilities.

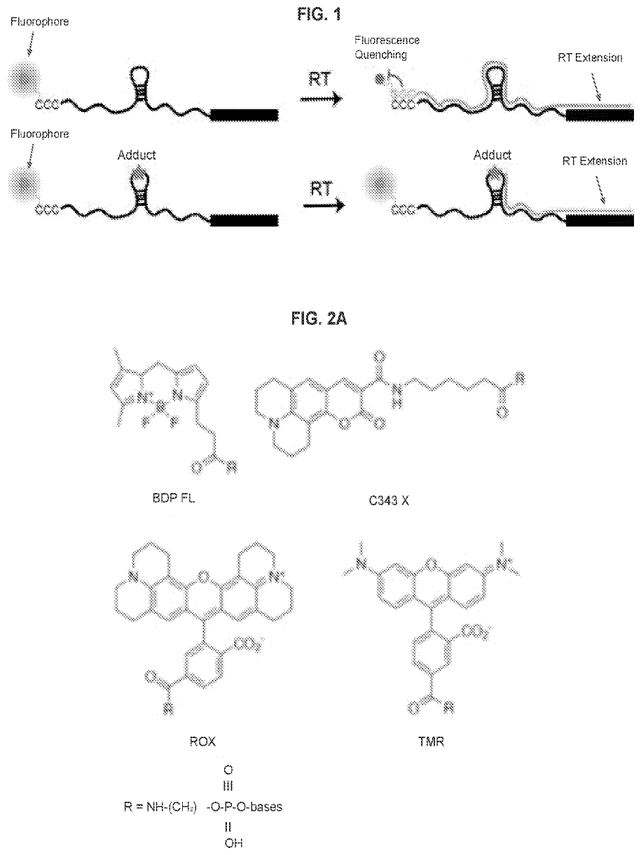

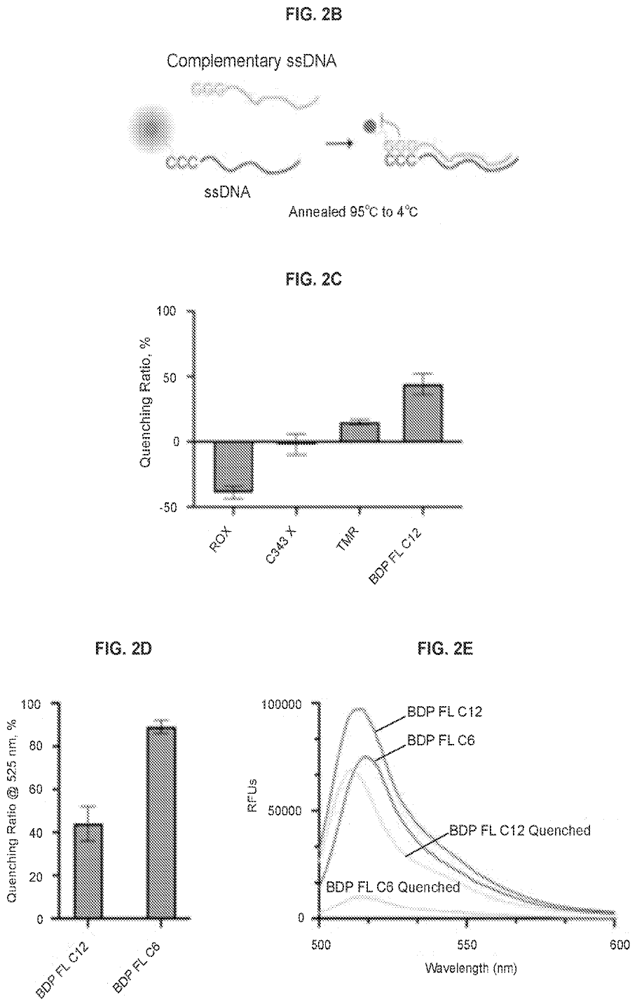

Bodipy fl (BDP-FL), tetramethylrhodamine (TMR), and coumarin 343X (C343 X) were selected for their known ability to be quenched using single-stranded beacons (FIG. 2A). A ROX fluorophore was also utilized as it is known to have increased fluorescence when in proximity to double stranded DNA upon hybridization. The 5′-end of the appended oligonucleotides was designed to be C-rich as it has been observed that hybridizing DNA beacons in which fluorophores are brought into proximity of G-rich ends results in efficient fluorescent quenching (FIG. 2B). This quenching is thought to be caused by a photoinduced electron transfer mechanism. The fluorophores were attached to the 5′-end of a single-stranded 5′-amino DNA oligonucleotide using NHS ester chemistry. Reactions contained excess NHS ester fluorophore, 30% NaHCO₃, 20% DMSO and were performed at room temperature overnight.

First, different fluorophores were tested for their ability to efficiently quench when hybridized to G-rich antisense oligonucleotides. As shown in FIG. 2C, incubation of fluorophore-conjugated DNA with G-rich antisense oligonucleotide resulted in varied quenching with BDP-FL, TMR, C343 X, and ROX. As predicted, hybridization resulted in an increase in ROX fluorescence. These results are consistent with other published reports demonstrating that BDP-FL fluorophores can be efficiently quenched by the interaction between itself and a guanine.

Having demonstrated that BDP-FL was quenched most efficiently, the linker distance between the C-rich 5′-end of the RNA molecule and the fluorophore was tested to determine if it could be shortened to enhance quenching by encouraging base-stacking between the BDP-FL fluorophore and the hybridized guanosine nucleotides. Upon replacing the 12-carbon spacer with a 6-carbon spacer, nearly 90% quenching when hybridized (FIG. 2D). This result is also demonstrated by looking at the fluorescent spectra showing a dramatic increase in overall quenching, and that quenching is observed for the entire fluorescent spectra when the shorter linker connecting the fluorophore with the oligonucleotide was utilized (FIG. 2E). These results demonstrate that BDP-FL fluorescence is efficiently quenched when hybridized with a G-rich oligonucleotide and that a shorter (6-carbon) linker increases the quenching.

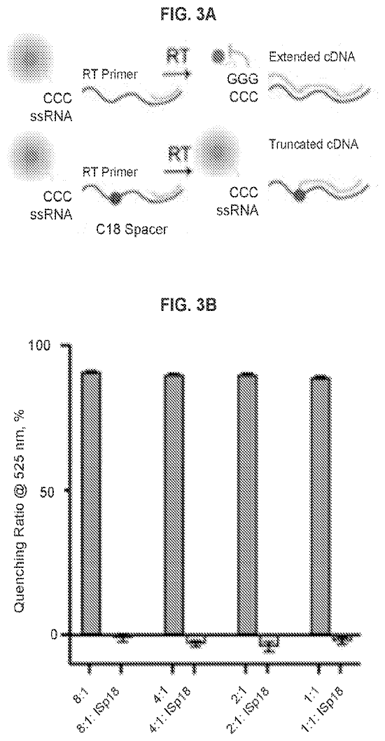

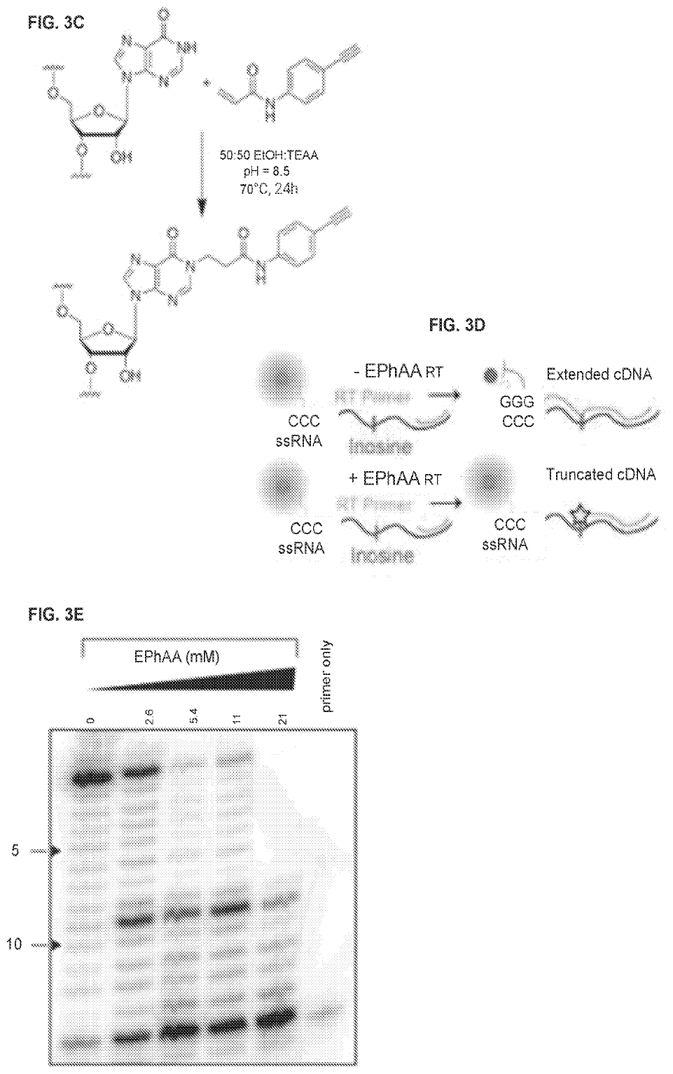

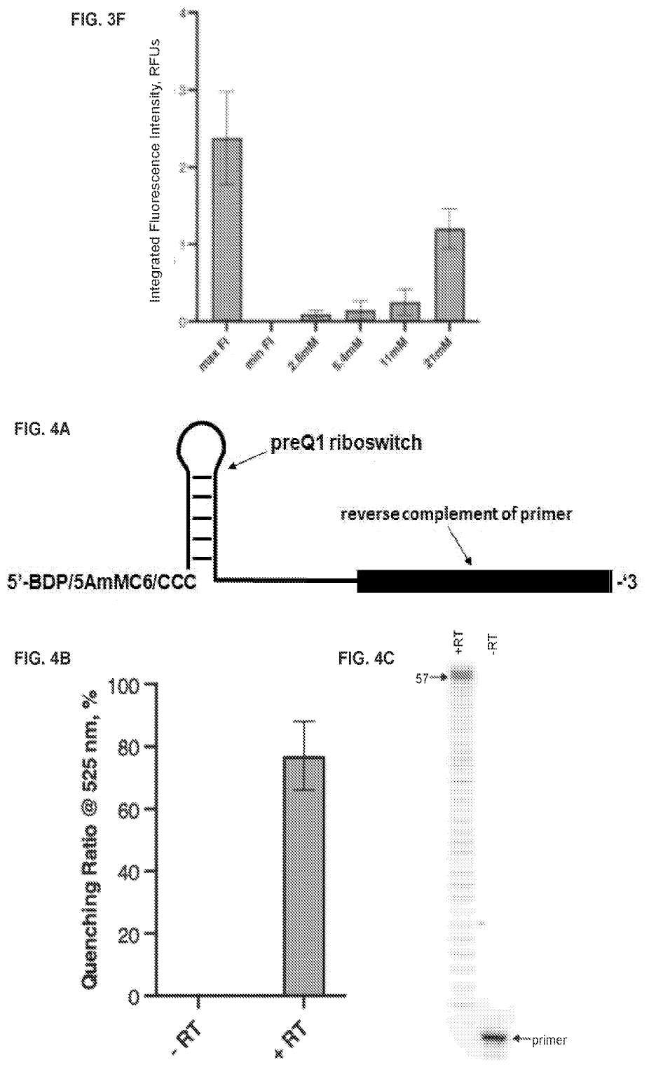

Having optimized the conditions for hybridization that enable efficient nucleotide-based quenching, the RT-based assay was the next focus. First, the conditions used for normal RT extension as well as those that would produce a truncated RT product were determined. As shown in FIG. 3A, an internal C18 spacer (labeled iSp18) was used, which is known to inhibit other processive enzymes, but had yet to be demonstrated with RT. Single-stranded RNA with increasing amounts of RT primer were incubated to determine the equivalents necessary to efficiently extend. As shown in FIG. 3B, efficient quenching in conditions ranging from 1:1-8:1 molar equivalents (RT Primer: RNA). It also observed that RNA with an iSp18 spacer had undetectable quenching, suggesting that the iSp18 spacer is a good control for halting RT processivity. Overall, these experiments demonstrated that the approach is amenable to fluorescent quenching using an RT assay and that the assay is able to detect full length RT processivity and truncations due to altered RT extension.

It was also desired to understand if the assay described herein would be sensitive enough to detect adducts formed between an RNA and an electrophilic compound. The goal is to utilize this approach to identify sites of RNA adduct formation through a reaction with a small molecule. To demonstrate this, the known reactivity between inosine and aryl acrylamide reagents was utilized. Inosine formation is a naturally occurring post-transcriptional modification that can control RNA function through re-coding open reading frames or altering RNA processivity. Inosine's unique chemical structure makes it amenable to reactivity with electrophilic small molecules with high selectivity.

Determining where inosine modifications occur is crucial to understanding how it relates to disease. Further, Inosine's unique chemical properties make it reactive to electrophilic small molecule compounds. It is thought that the unique chemical reactivity of inosine would make it amenable to small molecule induced adduct formation for potential therapeutic benefits. As such, the specific chemical reactivity of inosine was used as a positive control for assay development.

The known aryl acrylamide compound N-(4-ethynylphenyl) acrylamide (EPhAA) was used, which is an easy to synthesize aryl acrylamide that has been demonstrated to form adducts selectively with inosine. (FIG. 3C). We rationalized that this EPhAA adduct would result in blocking the W-C face of inosine, resulting in truncated RT products (FIG. 3D). Consistent with this concept, incubation of RNA with increasing molar excess of EPhAA resulted in the formation of truncated RT products as identified by denaturing gel electrophoresis (FIG. 3E). Reduction in fluorescent quenching in the RT assay using the same molar equivalents (FIG. 3F). Overall, these results strongly convey that RT truncations were able to be detected using our RT assay, and have demonstrated that this approach could be used to identify RNA adducts with an RT-based platform.

Herein the present invention has demonstrated the utility of RT-based reactions for fluorogenic detection of adducts between RNA and a small molecule. Fluorescence-based assays have been demonstrated to be widely used in drug discovery and basic biology, but there are limited examples of their applications for high-throughput discovery of RNA-small molecule interactions. Without wishing to limit the present invention to any theory or mechanism, it is believed that RT-based assays could pave the way for high-throughput measurements to detect these adducts in RNA, irregardless of the primary sequence. In future work RT-based assays will be further assessed for RNA adduct detection in the drug discovery sphere.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A method of detecting a potential adduct on a target oligonucleotide, the method comprising:
a) conjugating a fluorophore to the target oligonucleotide, wherein the fluorophore is downstream of the potential adduct to be detected; and
b) reverse transcribing (RT) the target oligonucleotide upstream of the potential adduct; wherein reverse transcribing forms an RT-extension;
wherein if no adduct exists on the target oligonucleotide, then the RT-extension quenches the fluorophore's fluorescence;
wherein if an adduct exists on the target oligonucleotide, then the RT-extension is truncated and the fluorophore's fluorescence is maintained.

2. The method of claim 1, wherein the fluorophore comprises both intrinsic and extrinsic fluorophores that would have their fluorescence altered by their proximity to nucleic acids.

3. (canceled)

4. The method of claim 1, wherein the fluorophore comprises Bodipy fl (BDP-FL), tetramethylrhodamine (TMR), coumarin 343X (C343 X), or a combination thereof.

5. The method of claim 1, wherein the fluorophore is conjugated to the target oligonucleotide via a linker.

6. The method of claim 5, wherein the linker comprises a chain having 3-15 carbons.

7. (canceled)

8. The method of claim 5, wherein the linker comprises one or more non-carbon, wherein the one or more non-carbons include oxygen, sulfur, phosphorus, and/or nitrogen.

9. The method of claim 1, wherein the target oligonucleotide comprises RNA or DNA.

10. (canceled)

11. The method of claim 1, wherein the_target oligonucleotide comprises single stranded RNA or single stranded DNA capable of forming more complex 2-D or 3-D structures.

12. The method of claim 1, where the adduct is a post-translational modification component.

13. The method of claim 12, wherein the post-translational component comprises N1-alkylation of inosine.

14. The method of claim 1, wherein detecting a potential adduct on a target oligonucleotide is used for high throughput screening.

15. A system for detecting an adduct on a target oligonucleotide, the system comprising:
a) a target oligonucleotide labeled with a fluorophore downstream of a potential adduct to be detected; and
b) a reverse transcribing (RT) component for acting upstream of the potential adduct; wherein the RT component reverse transcribes the target oligonucleotide to form an RT-extension;
i. if no adduct exists on the target oligonucleotide, then the RT-extension quenches the fluorophore's fluorescence;
ii. if an adduct exists on the target oligonucleotide, then the RT-extension is truncated and the fluorophore's fluorescence is maintained.

16. The system of claim 15, wherein the fluorophore comprises both intrinsic and extrinsic fluorophores that would have their fluorescence altered by their proximity to nucleic acids.

17. (canceled)

18. The system of claim 15, wherein the fluorophore comprises Bodipy fl (BDP-FL), tetramethylrhodamine (TMR), coumarin 343X (C343 X), or a combination thereof.

19. The system of claim 15, wherein the fluorophore is conjugated to the target oligonucleotide via a linker.

20. The system of claim 19, wherein the linker comprises a chain having 3-15 carbons.

21. (canceled)

22. The system of claim 19, wherein the linker comprises one or more non-carbons, wherein the one or more non-carbons include oxygen, sulfur, phosphorus, and/or nitrogen.

23. The system of claim 15, wherein the target oligonucleotide comprises RNA or DNA.

24. (canceled)

25. The system of claim 15, wherein the_target oligonucleotide further comprises single stranded RNA or single stranded DNA capable of forming more complex 2-D or 3-D structures.

26. The system of claim 15, wherein detecting a potential adduct on a target oligonucleotide is used for high throughput screening.
